(19) United States
(12) Patent Application Publication
(10) Pub. No.: US 2010/0057071 A1
(43) Pub. Date: Mar. 4, 2010

(54) ELECTROSURGICAL INSTRUMENT AND SYSTEM

(76) Inventors: Francis AMOAH, Berkshire (GB); Neil B. PARK, Berkshire (GB)

Correspondence Address: OSTROLENK FABER GERB & SOFFEN, 1180 AVENUE OF THE AMERICAS, NEW YORK, NY, 100368403, US

(21) Appl. No.: 12/546,835

(22) Filed: Aug. 25, 2009

Related U.S. Application Data
(60) Provisional application No. 61/136,489, filed on Sep. 9, 2008.

(30) Foreign Application Priority Data
Aug. 26, 2008 (GB) 0815515.2

(51) Int. Cl. A61B 18/18

(52) U.S. Cl. 606/33

(57) ABSTRACT

An electrosurgical system for cutting and coagulating tissue includes a generator for generating radio frequency (RF) power, and an electrosurgical instrument. The instrument includes a pair of blades pivotally joined for relative movement in a scissors-like action between open and closed positions, each of said blades including a conductive blade member and a conductive electrode electrically isolated from the blade member by means of an insulation member. A first one of either the conductive blade member or the conductive electrode constitutes an inner electrode on each blade, which are disposed in face-to-face relationship. A second one of either the conductive blade member or the conductive electrode constitutes an outer electrode on each blade spaced from the inner electrode. The instrument further includes an actuation mechanism for effecting relative movement of the blades in said scissors-like action, the generator including at least one source of RF power and a controller such that the generator is capable of delivering a first cutting RF waveform to the electrosurgical instrument or a second coagulating RF waveform to the electrosurgical instrument. The system further includes a switching circuit such that the first cutting RF waveform is delivered between the inner and outer electrodes on each blade, and the second coagulating RF waveform is delivered between the outer electrodes on each blade.

### CROSS-REFERENCE TO RELATED APPLICATION

This application claims the benefit of Provisional Application No. 61/136,489 filed Sep. 9, 2008.

### FIELD OF THE INVENTION

This invention relates to an electrosurgical system comprising a generator and an electrosurgical instrument including electrosurgical electrodes for receiving radio frequency (RF) power from the generator. Such systems are commonly used for the cutting and/or coagulation of tissue in surgical intervention, most commonly in “keyhole” or minimally invasive surgery, but also in laparoscopic and “open” surgery.

### BACKGROUND OF THE INVENTION

Electrosurgical instruments commonly known as a bipolar scissors instruments are disclosed in U.S. Pat. No. 6,179,837 and U.S. Reissue Pat. No. Re 36,795, although many other examples are available. Such devices use a radio frequency (RF) electrosurgical voltage to coagulate tissue, and the mechanical shearing action of the scissor blades to cut the tissue. The present invention attempts to provide an improvement to this type of instrument.

### SUMMARY OF THE INVENTION

According to the invention, an electrosurgical system for cutting and coagulating tissue includes a generator for generating radio frequency (RF) power, and an electrosurgical instrument including a pair of blades pivotally joined for relative movement in a scissors-like action between open and closed positions. Each of the blades includes a conductive blade member and a conductive electrode electrically isolated from the blade member by means of an insulation member, a first one of either the conductive blade member or the conductive electrode constituting an inner electrode on each blade. The inner electrodes are disposed in face-to-face relationship. A second one of either the conductive blade member or the conductive electrode constitutes an outer electrode on each blade spaced from the inner electrode. The instrument further includes an actuation mechanism for effecting relative movement of the blades in the scissors-like action. The generator includes at least one source of RF power and a controller such that the generator is capable of delivering a first cutting RF waveform to the electrosurgical instrument or a second coagulating RF waveform to the electrosurgical instrument, the system further including circuitry arranged to cause the first cutting RF waveform to be delivered between the inner and outer electrodes on each blade, and the coagulating RF waveform to be delivered between the outer electrodes of the respective blade.

There are examples of instruments in which an electrosurgical cutting signal is used to cut as well as coagulate tissue, but such instruments are generally forceps instruments. As stated previously, in scissors instruments the scissor blades are used to cut the tissue. U.S. Pat. No. 6,174,309 and U.S. Pat. No. 7,204,835 are examples of forceps instruments with an electrosurgical cut.

In contrast, disclosed herein is an electrosurgical scissors instrument, which, unlike a forceps instrument, does provide the option for a mechanical cut using the scissor blades. In addition, the system provides a bipolar electrosurgical cutting option, which has safety and control advantages when compared with a monopolar electrosurgical action.

In one embodiment of the present invention, the instrument relies solely on the electrosurgical cutting of tissue, and the shearing edge of one or both blades is preferably rounded so as to discourage the mechanical cutting of tissue. In this way, the user of the instrument can use the instrument with confidence, knowing that tissue will only be severed when the electrosurgical cut voltage is supplied, not by any movement of the instrument.

The electrosurgical instrument is configured differently in order to be able to produce an electrosurgical cut. The insulation member separating the inner and outer electrodes of each blade is preferably such that the separation between the electrodes is at least 0.2 mm. This may be achieved by the thickness of the insulation member being at least 0.2 mm, or by offsetting one of the electrodes such that the spacing is at least this amount. Preferably, the ratio of the cross-sectional thickness of the outer electrodes to the cross-sectional thickness of the inner electrodes is at least 2:1, and the cross-sectional thickness of the outer electrodes is at least 0.3 mm.

The generator is such that the first cutting waveform delivered between the inner and outer electrodes on each blade is conveniently at least 200 volts peak, preferably at least 300 volts peak, and typically in the range 300 to 500 volts peak. In one convenient arrangement, the generator has a controller that allows the generator to deliver a blended waveform consisting of a rapidly alternating combination of the cutting RF waveform and the coagulating RF waveform. Such a blended signal is disclosed in our U.S. Pat. No. 6,966,907, the details of which are hereby incorporated by reference.

In one convenient arrangement, at least one of the blades has a weak section such that the blade can flex laterally so as to increase the lateral distance between the blades when tissue is present therebetween. This has the advantage that a larger amount of tissue can be accommodated between the blades, and also allows the blades to press the opposite walls of skeletonized tissues such as vessels one against the other. It has been found that tissue sealing is effectively accomplished by pressing together the opposite walls of vessels, and applying heat to seal them one against the other. Conveniently, both blades are provided with a weak section such that both blades can flex laterally so as to increase the lateral distance between the blades when tissue is present therebetween. Typically, the or each weak section is such that the force needed to produce a flexing of the or each blade is at a first lower level when the deflection is below a predetermined threshold distance, and at a second higher level when the deflection is above the predetermined distance. The or each weak section is conveniently produced by having a recess or cut-out in the blade in the region between the area in which the blades contact tissue, and the area in which the blades pivot, so that a portion of reduced thickness is created. The recess is conveniently shaped to give the preferential flexibility up to the predetermined threshold blade separation.

The invention also resides in an electrosurgical instrument for cutting and coagulating tissue, the instrument including a pair of blades pivotally joined for relative movement in a scissors-like action between open and closed positions, each of said blades including a conductive blade member and a conductive electrode electrically isolated from the blade member by means of an insulation member, a first one of either the conductive blade member or the conductive electrode constituting an inner electrode on each blade. The inner electrodes are disposed in face-to-face relationship to provide a shearing edge therebetween, and a second one of either the conductive blade member or the conductive electrode constitutes an outer electrode on each blade spaced from the inner electrode. The instrument further includes an actuation mechanism for effecting relative movement of the blades in said scissors-like action, the shearing edge of one or both blades being rounded so as to discourage the mechanical cutting of tissue. The insulation member separates the inner and outer electrodes of each blade by a distance of at least 0.2 mm such that the instrument is capable of cutting tissue not by mechanical shearing but by means of a cutting RF waveform delivered between the inner and outer electrodes.

As before, the ratio of the cross-sectional thickness of the outer electrodes to the cross-sectional thickness of the inner electrodes is preferably at least 2:1, and the cross-sectional thickness of the outer electrodes is at least 0.3 mm. Also as previously described, the or each blade can be provided with a weak section to permit flexing of the or each blade to increase the lateral distance between the blades.

The invention further resides in an electrosurgical system for cutting and coagulating tissue, the system including a generator for generating radio frequency (RF) power, and an electrosurgical instrument including a pair of blades pivotally joined for relative movement in a scissors-like action between open and closed positions, wherein a first one of said blades includes a first electrode and a second electrode electrically isolated one from the other by means of an insulation member, and a second one of the blades including a third electrode, the instrument further including an actuation mechanism for effecting relative movement of the blades in said scissors-like action, and wherein the generator includes at least one source of RF power and a controller arranged such that the generator is capable of delivering a first, cutting RF waveform to the electrosurgical instrument or a second, coagulating RF waveform to the electrosurgical instrument, the system further including circuitry arranged to cause the cutting RF waveform to be delivered between the first and second electrodes, and the coagulating RF waveform to be delivered between one of the first and second electrodes and the third electrode.

The first and second electrodes are preferably located transversely as inner and outer electrodes, or alternatively located side by side on the blade with the insulating member therebetween. The second blade may be a unitary blade constituting the third electrode, or may contain additional electrodes in a sandwich or side by side structure.

The invention will be further described below, by way of example only, with reference to the accompanying drawings.

### BRIEF DESCRIPTION OF THE DRAWINGS

In the drawings:

FIG. 1 is a schematic diagram of an electrosurgical system in accordance with the invention,

FIG. 2 is a schematic plan view of the distal portion of a scissors-type instrument for use in the system of FIG. 1,

FIG. 3 is a schematic diagram of the output stage of a generator forming part of the system of FIG. 1,

FIG. 4 is a diagrammatic cross-section of the blades of the scissors-type instrument of FIG. 2, shown in use and in a first orientation to perform the coagulation of tissue,

FIG. 5 is a diagrammatic cross-section of the blades, shown in a second orientation to perform the coagulation of tissue,

FIG. 6 is a diagrammatic cross-section of the blades, shown in a first orientation to perform the cutting of tissue,

FIGS. 7 and 8 are plan views of the distal portion of an alternative scissors-type instrument in accordance with the invention,

FIG. 9 is a plan view of the distal portion of a further scissors-type instrument,

FIG. 10 is a diagrammatic cross-section of the blades of the scissors-type instrument of FIG. 9, shown in use,

FIGS. 11 and 12 are plan views of the distal portion of yet a further scissors-type instrument in accordance with the invention, and

FIGS. 13 and 14 are diagrammatic cross-sections of the blades of alternative embodiments of scissors-type instruments in accordance with the invention.

### DETAILED DESCRIPTION OF THE PREFERRED EMBODIMENTS OF THE INVENTION

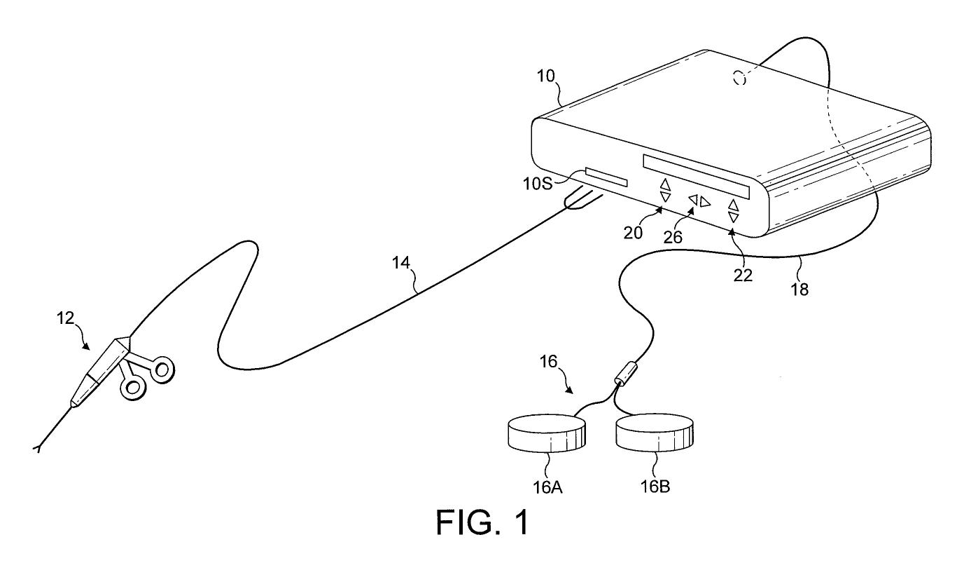

Referring to FIG. 1, a generator 10 has an output socket 10S providing a radio frequency (RF) output for an instrument 12 via a connection cord 14. Activation of the generator may be performed from the instrument 12 via a connection in cord 14 or by means of a footswitch unit 16, as shown, connected to the rear of the generator by a footswitch connection cord 18. In the illustrated embodiment footswitch unit 16 has two footswitches 16A and 16B for selecting a coagulation mode and a cutting mode of the generator respectively. The generator front panel has push buttons 20 and 22 for respectively setting coagulation and cutting power levels, which are indicated in a display 24. Push buttons 26 are provided as a means for selection between alternative coagulation and cutting waveforms.

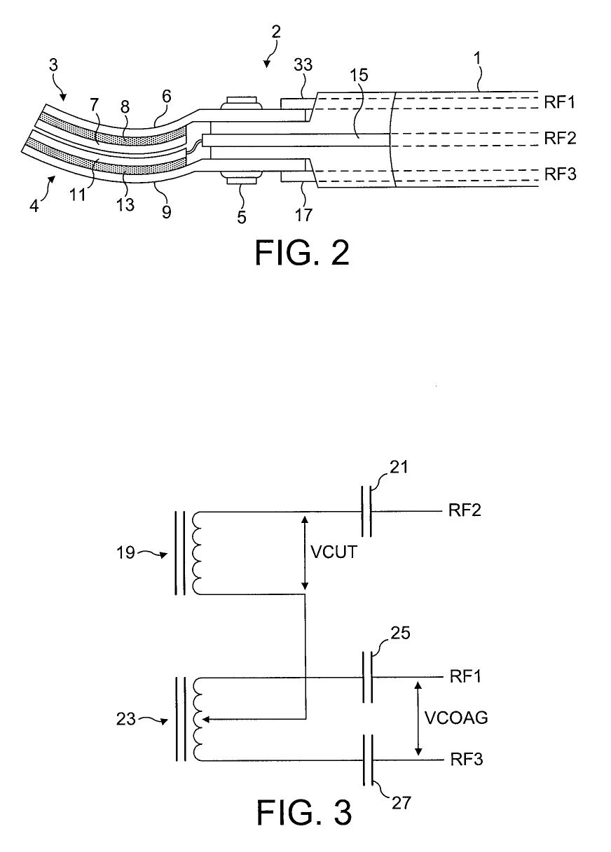

FIG. 2 shows an embodiment of the instrument 12 in more detail. The instrument 12 is a bipolar scissors device including an elongated tubular shaft 1 with a distal end 2, containing first and second scissors blades 3 and 4. The blades are actuated by a conventional handle assembly (not shown), which causes the blades to pivot about a pivot pin 5 between open and closed positions.

The first blade 3 comprises a conductive blade body 6 constituting a first electrode. The blade body 6 carries a second electrode 7 spaced from the blade body by an insulating spacer 8, typically of a ceramic material. The second blade 4 is of a similar construction, with a blade body 9 forming a first electrode, and a second electrode 11 separated from the blade body by a ceramic insulator 13. The two blade bodies 6 and 9 are the components moved by the actuating mechanism to open and close the blades, and the second electrodes 7 and 11 lie in face-to-face juxtaposition when the blades are in their closed position. Unlike those of a conventional scissors instrument, the edges of the second electrodes are rounded where they overlap so that they do not cause the mechanical cutting of tissue positioned between the blades when the blades are closed.

A first conductive actuation rod 33 is conductively connected to the blade body 6, and extends back along the instrument to be connected to an output RF1 of the electrosurgical generator 10. A lead 15 is connected to the second electrode 11 of blade 4, and extends back along the instrument to be connected to a second output RF2 of the electrosurgical generator 10. Finally, a second conductive actuation rod 17 is conductively connected to the blade body 9 and extends back along the instrument to be connected to a third output RF3 of the electrosurgical generator 10.

The output stage of the generator 10 is shown in FIG. 3. A first output transformer 19 is dedicated to providing a cut voltage via a coupling capacitor 21 between output connection RF2 and the midpoint of a second transformer 23. The second transformer 23 is dedicated to providing a coagulating voltage via coupling capacitors 25 and 27 between output connections RF1 and RF3. The cut or coagulation voltage signals are selected by a controller (not shown), as previously described in our U.S. Pat. No. 6,984,231 or 6,966,907. In one arrangement either the cut or the coagulation output is selected by the controller. In the other arrangement, the cut and coagulation outputs are supplied as a blended signal, with the cut and coagulation outputs being supplied in a rapidly alternating manner, i.e. alternating between one and then the other. The cut voltage, between output connections RF2 and the other two output connections, is typically in the range of 300 to 500 volts peak, while the coagulation voltage, between outputs RF1 and RF3 is typically less than 170 volts peak.

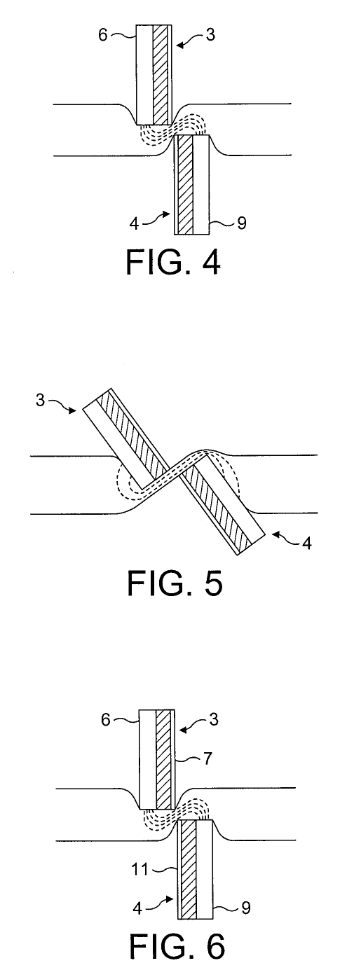

The operation of the electrosurgical system will now be described with reference to FIGS. 4 to 6. FIG. 4 shows the scissor blades 3 and 4 contacting tissue, and a coagulating voltage being supplied from the generator 10 between the output connections RF1 and RF3. This causes the coagulating voltage to be supplied between the blade bodies 6 and 9 of the respective blades 3 and 4. The RF current flows through the tissue from one blade body to the other, causing the tissue between the blades to be coagulated. Tilting the instrument 12, to cause the blades 3 and 4 to be rotated about their longitudinal axis, as shown in FIG. 5, results in the further coagulation of the tissue stretched between the blades.

In FIG. 6 the blades are reoriented to contact the now coagulated tissue, and the cutting voltage is supplied by the generator between output connection RF2 and the other two output connections. This causes the cutting voltage to be supplied to the second electrode 11 on the second blade 4, and also to the second electrode 7 on the first blade 3 by virtue of the second electrodes 7 and 11 being in contact with each other where they overlap towards the pivot pin 5. The second electrodes therefore act as the active electrode of a bipolar cutting assembly, with the blade bodies 6 and 9 acting as the return electrodes for the bipolar assembly. RF current passes through the tissue between the first and second electrodes of each blade, and, to a lesser extent, between the first electrode of one blade and the second electrode of the other blade. The RF current density is highest in tissue directly in contact with each of the second electrodes and this focuses the total delivered RF power to this tissue region. The local dissipation results in local evaporation of intercellular electrolytes and the progressive loss of electrical contact between the electrodes and tissue further focuses the delivered RF power to the remaining points of electrical contact. As electrolyte medium electrical contact is lost between the electrodes and the tissue, electrical arcs are drawn due to the high amplitude of applied cut voltage. These arcs propagate into a plasma enveloping the electrodes and the immediately adjacent tissue. This plasma causes the tissue to be severed in the region of the blades 3 and 4, although it is the plasma that severs the tissue as opposed to the mechanical shearing action of the closure of the blades. This means that the cutting action is more closely controlled, as it only commences when the electrosurgical generator 10 is activated, not by any mechanical movement of the blades 3 & 4.

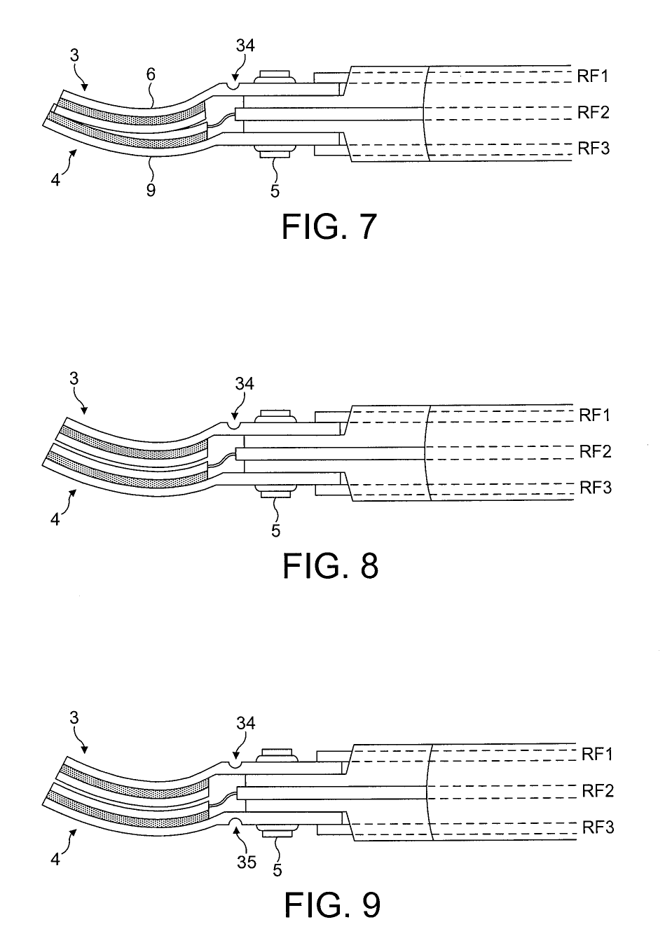

FIGS. 7 and 8 show first and second blades 3 and 4 similar to those previously described with reference to FIG. 1, but with the first blade 3 having a resilient or weak section as a result of and in the region of a recess 34 present on the blade body 6 in the region of the pivot pin 5. Notice how in FIG. 7 the blades 3 and 4 overlap one with the other towards the distal end of the blades, whereas in FIG. 8 the blade 3 has deflected due to the presence of tissue (not shown) between the blades such that the blades no longer overlap, and are slightly separated with thin tissue therebetween. It is expected that the separation will be in the order of between 0.05 mm and 0.2 mm under normal circumstances.

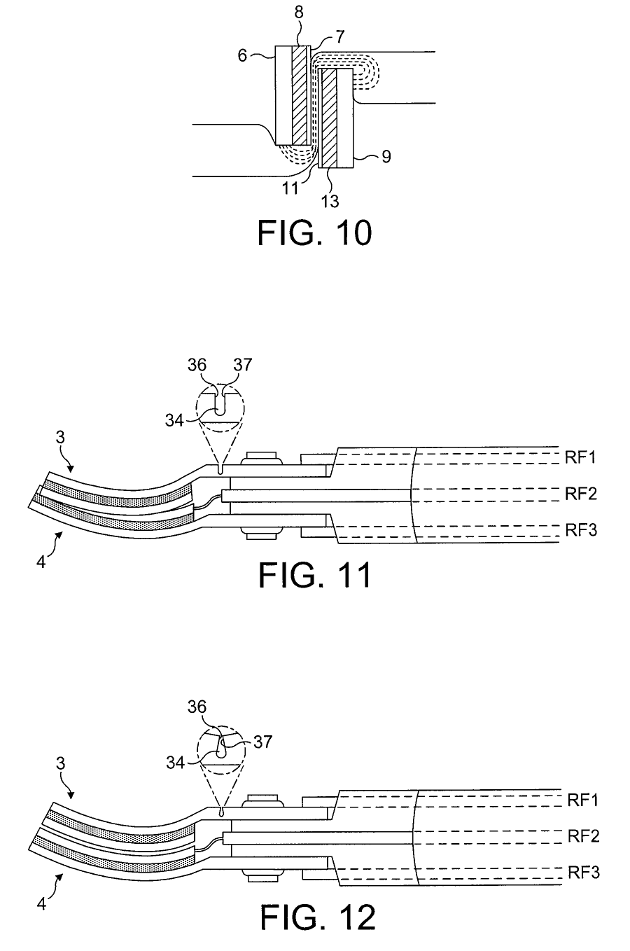

FIG. 9 shows an instrument in which recesses 34 and 35 are provided in both blades 3 and 4, recess 35 being provided in the second blade 4 to match the recess 34 previously described with reference to the first blade 3. This allows both blades 3 and 4 to deflect resiliently when tissue is accommodated therebetween. FIG. 10 shows how the tissue is trapped between the blades 3 and 4, which deflect to allow the tissue to be received between the blades without being mechanically cut. Trapping the tissue in this way and allowing the blades to deflect permits a controlled pressure to be applied to the tissue by the blades during coagulation thereof.

FIGS. 11 and 12 show how the shape of the recesses 34 and 35 can be used to control the deflection of the blades. In FIG. 11 the blades 3 and 4 deflect relatively easily, as the side wall 36 of each recess can move towards the opposite side wall 37, closing the gap therebetween. Once a predetermined level of deflection of the blades has occurred, the side walls are in contact with each other, as shown in FIG. 12. In this situation further deflection of the blades 3 & 4 is discouraged, and only takes place if a much larger deflection force is applied to the blades. This arrangement ensures that a relatively controlled force is applied to the tissue trapped between the blades, which has been found to produce good coagulation of the tissue.

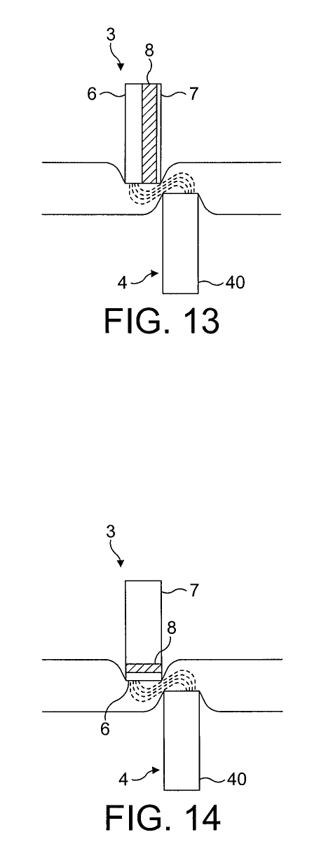

FIG. 13 shows an arrangement in which the first blade 3 comprises the blade body/first electrode 6 and the second electrode 7 spaced apart by the insulating spacer 8, as previously described. However, unlike in the arrangement of FIGS. 4 to 6, in which the second blade 4 is of a similar construction, the second blade in this embodiment comprises a single unitary metallic blade constituting a third electrode 40. When a user of the instrument wants to cut tissue, the cutting voltage from the generator is supplied between the first and second electrodes 6, 7, as before. When a user of the instrument wants to coagulate tissue, the coagulating voltage from the generator is supplied between the first electrode 6 and the third electrode 40. Alternatively, the coagulating voltage can be supplied between the second electrode 7 and the third electrode 40, or conceivably between both the first and second electrodes 6, 7 and the third electrode 40. This is a simpler construction because only one of the blades requires the sandwich structure with multiple electrodes on a single blade.

FIG. 14 shows an arrangement in which the first and second electrodes 6, 7 are present on the first blade 3, and with a unitary second blade 4. However in this construction, the first and second electrodes 6, 7 are provided in a side-by-side arrangement with the insulating member 8 therebetween, and with the insulating member 8 extending from one face of the blade 3 to the other rather than generally parallel to the faces. It will be noted that in this variant, both the first and second electrodes 6, 7, and the insulation member 8, are exposed on the inner face of the first blade 3, i.e. the face which faces the second blade 4 in the closed position. As before, cutting of tissue is effected by supplying a cutting voltage from the generator between the first and second electrodes 6, 7, while coagulation is established by supplying a coagulating voltage between the first and third electrodes 6, 40, between the second and third electrodes 7, 40, or conceivably between (a) both the first and second electrodes 6, 7 and (b) the third electrode 40.

Generally, it will be appreciated that other embodiments of the generator and the blade members can be envisioned without departing from the scope of the present invention. As described earlier, the electrodes on one or each blade may be provided in a layered sandwich construction, in a side-by-side construction, or even in a staggered construction containing elements of each previous arrangement. By employing an electrosurgical cutting signal to sever the tissue, as opposed to the mechanical shearing between the blades, a more controlled cut can be obtained.

1. An electrosurgical system for cutting and coagulating tissue, the system including a generator for generating radio frequency (RF) power, and an electrosurgical instrument including a pair of blades pivotally joined for relative movement in a scissors-like action between open and closed positions, wherein each of said blades includes a conductive blade member and a conductive electrode electrically isolated from the blade member by means of an insulation member, a first one of either the conductive blade member or the conductive electrode constituting an inner electrode on each blade, the inner electrodes being disposed in face-to-face relationship, and a second one of either the conductive blade member or the conductive electrode constituting an outer electrode on each blade spaced from the inner electrode, the instrument further including an actuation mechanism for effecting relative movement of the blades in said scissors-like action, and wherein the generator includes at least one source of RF power and a controller arranged such that the generator is capable of delivering a first, cutting RF waveform to the electrosurgical instrument or a second, coagulating RF waveform to the electrosurgical instrument, the system further including circuitry arranged to cause the cutting RF waveform to be delivered between the inner and outer electrodes on each blade, and the coagulating RF waveform to be delivered between the outer electrodes of the respective blades.

2. An electrosurgical system according to claim 1, wherein the shearing edge of one or both blades is rounded so as to discourage the mechanical cutting of tissue.

3. An electrosurgical system according to claim 1, wherein the insulation member separating the inner and outer electrodes of each blade is such that the separation between the electrodes is at least 0.2 mm.

4. An electrosurgical system according to claim 1, wherein the ratio of the cross-sectional thickness of the outer electrodes to the cross-sectional thickness of the inner electrodes is at least 2:1.

5. An electrosurgical system according to claim 1, wherein the cross-sectional thickness of the outer electrodes is at least 3 mm.

6. An electrosurgical system according to claim 1, wherein the generator is such that the first, cutting waveform delivered between the inner and outer electrodes on each blade is at least 200 volts peak.

7. An electrosurgical system according to claim 6, wherein the generator is such that the first, cutting waveform delivered between the inner and outer electrodes on each blade is at least 300 volts peak.

8. An electrosurgical system according to claim 7, wherein the generator is such that the first, cutting waveform delivered between the inner and outer electrodes on each blade is in the range 300 to 500 volts peak.

9. An electrosurgical system according to claim 1, wherein the controller of the generator is arranged such that the generator is capable of delivering a blended waveform consisting of a rapidly alternating combination of the first, cutting RF waveform and the second, coagulating RF waveform.

10. An electrosurgical system according to claim 1, wherein at least one of the blades has a weak section such that the blade can flex laterally so as to increase the lateral distance between the blades when tissue is present therebetween.

11. An electrosurgical system according to claim 10, wherein both blades are provided with a weak section such that both blades can flex laterally so as to increase the lateral distance between the blades when tissue is present therebetween.

12. An electrosurgical system according to claim 10, wherein the or each weak section is formed such that the force needed to produce a flexing of the or each blade is at a first lower level when the deflection is below a predetermined threshold distance, and at a second higher level when the deflection is above the predetermined distance.

13. An electrosurgical instrument for cutting and coagulating tissue, the instrument including a pair of blades pivotally joined for relative movement in a scissors-like action between open and closed positions, wherein each of said blades includes a conductive blade member and a conductive electrode electrically isolated from the blade member by means of an insulation member, a first one of either the conductive blade member or the conductive electrode constituting an inner electrode on each blade, the inner electrodes being disposed in face-to-face relationship to provide a shearing edge therebetween, and a second one of either the conductive blade member or the conductive electrode constituting an outer electrode on each blade spaced from the inner electrode, the instrument further including an actuation mechanism for effecting relative movement of the blades in said scissors-like action, wherein the shearing edge of one or both blades is rounded so as to discourage the mechanical cutting of tissue, and wherein the insulation member separates the inner and outer electrodes of each blade by a distance of at least 0.2 mm such that the instrument is capable of cutting tissue not by mechanical shearing but by means of a cutting RF waveform delivered between the inner and outer electrodes.

14. An electrosurgical instrument according to claim 13, wherein the ratio of the cross-sectional thickness of the outer electrodes to the cross-sectional thickness of the inner electrodes is at least 2:1.

15. An electrosurgical instrument according to claim 13, wherein the cross-sectional thickness of the outer electrodes is at least 0.3 mm.

16. An electrosurgical instrument according to claim 13, wherein at least one of the blades has a weak section such that the blade can flex laterally so as to increase the lateral distance between the blades when tissue is present therebetween.

17. An electrosurgical instrument according to claim 16, wherein both blades are provided with a weak section such that both blades can flex laterally so as to increase the lateral distance between the blades when tissue is present therebetween.

18. An electrosurgical instrument according to claim 16, wherein the or each weak section is such that the force needed to produce a flexing of the or each blade is at a first lower level when the deflection is below a predetermined threshold distance, and at a second higher level when the deflection is above the predetermined distance.

19. An electrosurgical system for cutting and coagulating tissue, the system including a generator for generating radio frequency (RF) power, and an electrosurgical instrument including a pair of blades pivotally joined for relative movement in a scissors-like action between open and closed positions, wherein a first one of said blades includes a first electrode and a second electrode electrically isolated one from the other by means of an insulation member, and a second one of the blades includes a third electrode, the instrument further including an actuation mechanism for effecting relative movement of the blades in said scissors-like action, and wherein the generator includes at least one source of RF power and a controller arranged such that the generator is capable of delivering a first, cutting RF waveform to the electrosurgical instrument or a second, coagulating RF waveform to the electrosurgical instrument, the system further including circuitry arranged to cause the cutting RF waveform to be delivered between the first and second electrodes, and the coagulating RF waveform to be delivered between one of the first and second electrodes and the third electrode.

20. An electrosurgical system according to claim 19, wherein the first and second electrodes are located transversely as inner and outer electrodes.

21. An electrosurgical system according to claim 19, wherein the first and second electrodes are located side by side on the blade with the insulating member therebetween.

22. An electrosurgical system according to claim 2, wherein the insulation member separating the inner and outer electrodes of each blade is such that the separation between the electrodes is at least 0.2 mm.

23. An electrosurgical system according to claim 11, wherein the or each weak section is formed such that the force needed to produce a flexing of the or each blade is at a first lower level when the deflection is below a predetermined threshold distance, and at a second higher level when the deflection is above the predetermined distance.

24. An electrosurgical instrument according to claim 14, wherein the cross-sectional thickness of the outer electrodes is at least 0.3 mm.

25. An electrosurgical instrument according to claim 17, wherein the or each weak section is such that the force needed to produce a flexing of the or each blade is at a first lower level when the deflection is below a predetermined threshold distance, and at a second higher level when the deflection is above the predetermined distance.
